(19) United States
(12) Patent Application Publication
(10) Pub. No.: US 2025/0090464 A1
(43) Pub. Date: Mar. 20, 2025

(54) MAGNESIUM DIACETATE X 4H 2O AND/OR MAGNESIUM DICHLORIDE X 6H 2O AND/OR MAGNESIUM SULFATE X 7H 2O AND/OR DISODIUM SULFATE X 10 H 2O AND/OR SODIUM THIOSULFATE X 5H 2O AND/OR NA 3PO 4 X 12 H 2O AND/OR NA 2HPO 4 X 12H 2O/7H 2O AND/OR SODIUM ACETATE X 3H 2O AS GRANULATING AID

(71) Applicant: BASF SE, Ludwigshafen (DE)

(72) Inventors: Lukas Ries, Ludwigshafen (DE); Thorsten Cech, Ludwigshafen (DE); Elesabeth Gruenstein, Ludwigshafen (DE); Felicitas Guth, Ludwigshafen (DE); Karl Kolter, Limburgerhof (DE)

(21) Appl. No.: 18/727,804

(22) PCT Filed: Feb. 3, 2023
(86) PCT No.: PCT/EP2023/052657

§ 371 Date: Jul. 10, 2024

(30) Foreign Application Priority Data
Feb. 3, 2022 (EP) 22154889.4
Jun. 15, 2022 (EP) 22179206.2
Jan. 26, 2023 (EP) 23153457.9

(51) Int. Cl. A61K 9/16

(52) U.S. Cl. CPC A61K 9/1611; A61K 9/1694

(57) ABSTRACT

The present invention relates to a powder mixture for the preparation of a granulate comprising as constituent at least a filler or at least an active ingredient or both and as granulation aid Magnesium diacetate×4H₂O and/or Magnesium dichloride×6H₂O and/or Magnesium sulfate×7H₂O and/or Disodium sulfate xH₂O and/or Sodium thiosulfate×5H₂O and/or Na₃PO₄×12H₂O and/or Na₂HPO₄×12H₂O/7H₂O and/or Sodium acetate×3H₂O. Optionally constituents of the powder mixture to be granulated are binders, disintegrants and further customary auxiliaries. It was an object of the present invention to provide a powder mixture which can be granulated without adding a liquid but where the water for the granulation is released in-situ from Magnesium diacetate×4H₂O and/or Magnesium dichloride×6H₂O and/or Magnesium sulfate×7H₂O and/or Disodium sulfate xH₂O and/or Sodium thiosulfate×5H₂O and/or Na₃PO₄×12H₂O and/or Na₂HPO₄×12H₂O/7H₂O and/or Sodium acetate×3H₂O. An additional drying step is not required.

In the area of granulation, especially wet granulation, the addition of water to a raw powder mixture to guarantee particle growth through nucleation always results in a drying step. Drying of the wet granulates is expensive and requires resources such as energy, time, and machine utilization. With the market trend moving from batch processes towards continuous manufacturing this drying step is often performed through fluidized bed-drying, which is on the one hand expensive and on the other hand only a semi-continuous process, because one needs more fluidized bed machines to guarantee the continuity of the process. For a sustainable future a granulate manufactured by a process with reduced drying time and reduced energy to evaporate the water out of the wet granulates is needed. The wet granulation process according to the invention enables a wet granulation of a lot of excipients and active ingredients for which because of their moisture sensitivity the currently used wet granulation process is not suited to be granulated. This advantage in combination with shorter process times will result in a simplification of product development and manufacturing processes and supports the market trend for platform formulations. The platform formulation according to the invention is a powder mixture comprising an excipient that is capable releasing water during the granulation process in combination with a water-binding filler or a water binding active ingredient to reduce or preferably fully eliminate the need to add water as a granulation liquid and would make a drying step obsolete. The present invention relates to platform formulations permitting a granulation by applying thermal energy, such as heat, IR-waves, or microwaves to the powder mixture without adding any liquid. It was shown that Magnesium diacetate×4H₂O and/or Magnesium dichloride×6H₂O and/or Magnesium sulfate×7H₂O and/or Disodium sulfate×10H₂O and/or Sodium thiosulfate×5H₂O and/or Na₃PO₄×12 H₂O and/or Na₂HPO₄×12H₂O/7H₂O and/or Sodium acetate×3H₂O performed very good as a water releasing excipient enabling granulation of platform formulations (e.g., Na₂tartrate·2H₂O is not suitable for granulation at all). Through this energy application, it is possible to provide free water for nucleation and profit to the recrystallisation of salt resulting in dry granulates. It was unexpected that the resulting dry granulates were stable, nonhygroscopic, and very suitable for tableting. The resulting tablets have very high tensile strengths. In further experiments it could be shown that Magnesium diacetate×4H₂O and/or Magnesium dichloride×6H₂O and/or Magnesium sulfate×7H₂O and/or Disodium sulfate×10H₂O and/or Sodium thiosulfate×5H₂O and/or Na₃PO₄×12 H₂O and/or Na₂HPO₄×12H₂O/7H₂O and/or Sodium acetate×3H₂O can also be used for continuous water-free granulation of active ingredients (e.g., acetyl salicylic acid, paracetamol, ibuprofen). As these active ingredients are known to be difficult to formulate, the results were particularly surprising. besides, it could be used for all solid dosage forms like tablets, pellets, or granules.

The performance of Magnesium diacetate×4H₂O and/or Magnesium dichloride×6H₂O and/or Magnesium sulfate×7H₂O and/or Disodium sulfate×10H₂O and/or Sodium thiosulfate×5H₂O and/or Na₃PO₄×12 H₂O and/or Na₂HPO₄×12H₂O/7H₂O and/or Sodium acetate×3H₂O was particularly surprising, because other potentially water-releasing auxiliaries such as Alum, Calcium acetate, Calcium chloride, carrageenan, corn starch, cyclcodextrine, Kaolin, Magnesium citrate, Sodium citrate, Sodium sulfite Raffinose, rice starch, Tragacanth, Trehalose, Tricalcium citrate, wheat starch and Zinc sulfate did not lead to a formation of suitable granulates with the desired increase in particle size, or the properties of the resulting granulates were unfavorable sticky, hygroscopic or showed low compactability and yet others like Disodium sulfate×10H₂O, Magnesium sulfate×7H₂O, Magnesium dichloride×6H₂O and Na₂HPO₄×12H₂O/7H₂O are indeed technically suitable as a water releasing excipient for granulation purposes but are not applicable for pharmaceutical dosage forms anyway because of their taste or laxative effect on humans. Nonetheless the granulates could be used for agricultural, cosmetical, medical or pharmaceutical preparations, for food or feed, household cleaners, cleaning agents, laundry detergents, dishing washing tablets or mixtures thereof, with a coating if needed.

The present invention relates to a powder mixture for the preparation of a granulate comprising as constituent at least a filler or at least an active ingredient or both and as granulate Magnesium diacetate×4H₂O and/or Magnesium dichloride×6H₂O and/or Magnesium sulfate×7H₂O and/or Disodium sulfate×10H₂O and/or Sodium thiosulfate×5H₂O and/or Na₃PO₄×12 H₂O and/or Na₂HPO₄×12H₂O/7H₂O and/or Sodium acetate×3H₂O.

Optionally constituents of the powder mixture to be granulated are binders, disintegrants and further customary auxiliaries. Of course, it is also possible to add the at least one binder and/or at least one disintegrant and/or further customary auxiliaries in dry form to the granulates obtained. Object of the present invention is to provide a powder mixture which can be granulated without adding a liquid but where the water for the granulation is released in situ from Magnesium diacetate×4H₂O and/or Magnesium dichloride×6H₂O and/or Magnesium sulfate×7H₂O and/or Disodium sulfate×10H₂O and/or Sodium thiosulfate×5H₂O and/or Na₃PO₄×12 H₂O and/or Na₂HPO₄×12H₂O/7H₂O and/or Sodium acetate×3H₂O. An additional drying step is not required.

Accordingly, a powder mixture was found comprising a) 10-95% by weight of at least one filler, b) 2.5-10% by weight of at least one binder, c) 2.5-20% by weight of Magnesium diacetate×4H₂O and/or Magnesium dichloride×6H₂O and/or Magnesium sulfate×7H₂O and/or Disodium sulfate×10H₂O and/or Sodium thiosulfate×5H₂O and/or Na₃PO₄×12 H₂O and/or Na₂HPO₄×12H₂O/7H₂O and/or Sodium acetate×3H₂O, d) 0-7.5% by weight of at least one disintegrant and e) 0-85% by weight of at least one active ingredient the total of the constituents a) to e) being 100% by weight or a powder mixture was found comprising a) 10-95% by weight of at least one filler, b) 2.5-10% by weight of at least one binder, c) 2.5-20% by weight of Magnesium diacetate×4H₂O and/or Magnesium dichloride×6H₂O and/or Magnesium sulfate×7H₂O and/or Disodium sulfate×10H₂O and/or Sodium thiosulfate×5H₂O and/or Na₃PO₄×12 H₂O and/or Na₂HPO₄×12H₂O/7H₂O and/or Sodium acetate×3H₂O, d) 0-7.5% by weight of at least one disintegrant, e) 0-85% by weight of at least one active ingredient and f) 0-5% by weight further customary auxiliaries the total of the constituents a) to f) being 100% by weight.

The powder mixture comprises as constituent a) from 0 to 98.5% by weight, preferred from 7 to 97% by weight and more preferred from 10 to 95% by weight of a filler. Suitable fillers are, for example, lactose, wherein modified lactose or anhydrous (NF) lactose may be mentioned, starch, in particular modified (pregelatinized) starch, native starch or mixtures of both, calcium phosphate, in particular dibasic, unground dibasic and anhydrous dibasic calcium phosphate, cellulose derivatives, cellulose, in particular microcrystalline cellulose, mannitol, sorbitol, etc. Of course, mixtures of different fillers can be used.

Furthermore, the powder mixture comprises a binder as constituent b) in amounts of from 0 to 15% by weight, preferred from 1.5 to 10% by weight and more preferred from 2.5 to 10% by weight, of the total powder mixture. Suitable binders are water soluble polymers and excipients as well as film forming excipients for example polyvinylpyrrolidones, vinylpyrrolidone/vinylacetate Copolymers, polyvinyl alcohols, polyvinyl alcohols/polyethylene glycol graft copolymers, polyethylene glycols, ethylene glycol/propylene glycol block copolymers, hydroxypropylmethylcellulose, hydroxypropylcellulose, hydroxyethylcellulose, carrageenans, pectins, xanthans, lactose, sugar alcohols and alginates. Of course, also mixtures of different binders can be used.

Magnesium diacetate×4H₂O and/or Magnesium dichloride×6H₂O and/or Magnesium sulfate×7H₂O and/or Disodium sulfate×10H₂O and/or Sodium thiosulfate×5H₂O and/or Na₃PO₄×12 H₂O and/or Na₂HPO₄×12H₂O/7H₂O and/or Sodium acetate×3H₂O in amounts of from 1.5 to 30% by weight, preferred 2.5 to 25% by weight and more preferred 2.5 to 20% by weight is used as a granulation aid as constituent c) of the powder mixture. Magnesium diacetate×4H₂O and/or Magnesium dichloride×6H₂O and/or Magnesium sulfate×7H₂O and/or Disodium sulfate×10H₂O and/or Sodium thiosulfate×5H₂O and/or Na₃PO₄×12 H₂O and/or Na₂HPO₄×12H₂O/7H₂O and/or Sodium acetate×3H₂O with a mean particle size of about 500 μm can be used as constituent, but it was observed that Magnesium diacetate×4H₂O and/or Magnesium dichloride×6H₂O and/or Magnesium sulfate×7H₂O and/or Disodium sulfate×10H₂O and/or Sodium thiosulfate×5H₂O and/or Na₃PO₄×12 H₂O and/or Na₂HPO₄×12H₂O/7H₂O and/or Sodium acetate×3H₂O, with a mean particle size d (0.5) of less than 300 μm is particularly suitable as it improves the mixing behavior of the powder mixture.

Furthermore, the powder mixture may comprise as constituent d) disintegrants in amounts of from 0 to 12.5% by weight, preferred from 0 to 10% by weight, more preferred from 0 to 7.5%. Suitable disintegrants are crosslinked polyvinylpyrrolidone, croscarmellose, sodium starch glycolate also meaning according to the invention the sodium and potassium salts thereof. Furthermore, sodium carboxymethylstarch is suitable. Likewise suitable is L-hydroxypropylcellulose, preferably having 5 to 16% hydroxypropoxy groups. Of course, mixtures of different disintegrants can be used.

Optionally the powder mixture may also comprise 0 to 98.5% by weight, preferred from 0 to 90% by weight and more preferred from 0 to 85% by weight of an active ingredient as constituent e) of the powder mixture.

It is possible to employ as active ingredients in principle all active ingredients.

Examples of suitable active ingredients include, but are not limited to: analgesics and antiinflammatory drugs such as fentanyl, indomethacin, ibuprofen, naproxene, diclofenac, diclofenac sodium, fenoprofen, acetylsalicylic acid, ketoprofen, nabumetone, paracetamol, piroxicam, meloxicam, tramadol, and COX-2 inhibitors such as celecoxib and rofecoxib; antiarrhythmic drugs such as procainamide, quinidine and verapamil; antibacterial and antiprotozoal agents such as amoxicillin, ampicillin, benzathine penicillin, benzylpenicillin, cefaclor, cefadroxil, cefprozil, cefuroxime axetil, cephalexin, chloramphenicol, chloroquine, ciprofloxacin, clarithromycin, cla[1]vulanic acid, clindamycin, doxycycline, erythromycin, flucloxacillin sodium, halofantrine, isoniazid, kanamycin sul[1]phate, lincomycin, mefloquine, minocycline, nafcillin sodium, nalidixic acid, neomycin, nortloxacin, ofloxacin, oxa[1]cillin, phenoxymethyl-penicillin potassium, pyrimethaminesulfadoxime and streptomycin; anti-coagulants such as warfarin; antidepressants such as amitriptyline, amoxapine, butriptyline, clomipramine, desipramine, dothiepin, doxepin, fluox[1]etine, reboxetine, amineptine, selegiline, gepirone, imipramine, lithium carbonate, mianserin, milnacipran, nortriptyline, paroxetine, sertraline and 3-[2-[3,4-dihydrobenzofuro[3,2-c]pyridin-2 (1H)-yl]ethyl]-2-methyl-4H-pyrido[1,2-a]py[1]rimidin-4-one; anti-diabetic drugs such as glibenclamide and metformin; anti-epileptic drugs such as carbamazepine, clonazepam, ethosuximide, gabapentin, lamotrigine, levetiracetam, phenobarbitone, phenytoin, primidone, tiagabine, topiramate, valpromide and vigabatrin; antifungal agents such as amphotericin, clotrimazole, econazole, fluconazole, flucytosine, griseofulvin, itraconazole, ketoconazole, miconazole nitrate, nystatin, terbinafine and voriconazole; antihistamines such as astemizole, cinnarizine, cyproheptadine, decarboethoxyloratadine, fexofenadine, flunarizine, levocabastine, loratadine, norastemizole, oxatomide, promethazine and terfenadine; anti-hypertensive drugs such as captopril, enalapril, ketanserin, lisinopril, minoxidil, prazosin, ramipril, reserpine, terazosin and telmisartan; anti-muscarinic agents such as atropine sulphate and hyoscine; antineoplastic agents and antimetabolites such as platinum compounds, +such as cisplatin and carboplatin; taxanes such as paclitaxel and docetaxel; tecans such as camptothecin, irinotecan and topotecan; vinca alkaloids such as vinblastine, vindecine, vincristine and vinorelbine; nucleoside derivatives and folic acid antagonists such as 5-fluorouracil, capecitabine, gemcitabine, mercaptopurine, thioguanine, cladribine and methotrexate; alkylating agents such as the nitrogen mustards, e.g. cyclophosphamide, chlorambucil, chiormethine, iphosphamide, melphalan, or the nitrosoureas, e.g. carmustine, lomustine, or other alkylating agents, e.g. busulphan, dacarbazine, procarbazine, thiotepa; antibiotics such as daunorubicin, doxorubicin, idarubicin, epirubicin, bleomycin, dactinomycin and mito[1]mycin; podophyllotoxin derivatives such as etoposide and teniposide; famesyl transferase inhibitors; anthrachinon derivatives such as mitoxantron; anti-migraine drugs such as alniditan, naratriptan and sumatriptan; anti-Parkinsonian drugs such as bromocryptine mesylate, levodopa and selegiline; antipsychotic, hypnotic and sedating agents such as alprazolam, buspirone, chlordiazepoxide, chlorpromazine, clozapine, diazepam, flupenthixol, fluphenazine, flurazepam, 9-hydroxyrisperidone, lorazepam, mazapertine, olan[1]zapine, oxazepam, pimozide, pipamperone, piracetam, promazine, risperidone, selfotel, seroquel, sertindole, sulpir[1]ide, temazepam, thiothixene, triazolam, trifluperidol, ziprasidone and zolpidem; anti-stroke agents such as lubeluzole, lubeluzole oxide, riluzole, aptiganel, eliprodil and remacemide; antitussives such as dextromethorphan and laevodropropizine; antivirals such as acyclovir, ganciclovir, loviride, tivirapine, zidovudine, lamivudine, zidovudine/lamivudine, didano[1]sine, zalcitabine, stavudine, abacavir, lopinavir, amprenavir, nevirapine, efavirenz, delavirdine, indinavir, nelfinavir, ritonavir, saquinavir, adefovir and hydroxyurea; beta-adrenoceptor blocking agents such as atenolol, carvedilol, metoprolol, nebivolol and propanolol; cardiac inotropic agents such as amrinone, digitoxin, digoxin and milrinone; corticosteroids such as beclomethasone dipropionate, betamethasone, budesonide, dexamethasone, hydrocorti[1]sone, methylprednisolone, prednisolone, prednisone and triamcinolone; disinfectants such as chlorhexidine; diuretics such as acetazolamide, furosemide, hydrochlorothiazide and isosorbide; enzymes; gastro-intestinal agents such as cimetidine, cisapride, clebopride, diphenoxylate, domperidone, famotidine, lanso[1]prazole, loperamide, loperamide oxide, mesalazine, metoclopramide, mosapride, nizatidine, norcisapride, olsala[1]zine, omeprazole, pantoprazole, perprazole, prucalopride, rabeprazole, ranitidine, ridogrel and sulphasalazine; haemostatics such as aminocaproic acid; HIV protease inhibiting compounds such as ritonavir, lopinavir, indinavir, saquinavir, tipranavir; lipid regulating agents such as atorvastatin, fenofibrate, fenofibric acid, lovastatin, pravastatin, probucol and simv[1]astatin; local anaesthetics such as benzocaine and lignocaine; opioid analgesics such as buprenorphine, codeine, dextromoramide, dihydrocodeine, hydrocodone, oxycodone and morphine; parasympathomimetics and anti-dementia drugs such as eptastigmine, galanthamine, metrifonate, mil[1]ameline, neostigmine, physostigmine, tacrine, donepezil, rivastigmine, sabcomeline, talsaclidine, xanomeline, me[1]mantine and lazabemide; peptides and proteins such as antibodies, becaplermin, cyclosporine, tacrolimus, erythropoietin, immunoglobulins and insuline; sex hormones such as oestrogens: conjugated oestrogens, ethinyloestradiol, mestranol, oestradiol, oestriol, oestro[1]ne; progestogens; chlormadinone acetate, cyproterone acetate, 17-deacetyl norgestimate, desogestrel, dienogest, dydrogesterone, ethynodiol diacetate, gestodene, 3-keto desogestrel, levonorgestrel, lynestrenol, medroxyprogesterone acetate, megestrol, norethindrone, norethindrone acetate, norethisterone, norethisterone acetate, norethyn[1]odrel, norgestimate, norgestrel, norgestrienone, progesterone and quingestanol acetate; stimulating agents such as sildenafil, vardenafil; vasodilators such as amlodipine, buflomedil, amyl nitrite, diltiazem, dipyridamole, glyceryl trinitrate, isosorbide din[1]itrate, lidoflazine, molsidomine, nicardipine, nifedipine, oxpentifylline and pentaerythritol tetranitrate; their N-oxides, their pharmaceutically acceptable acid or base addition salts, their stereochemically isomeric forms, and their polymorphs Mixtures of active ingredients can also be employed.

Furthermore, the powder mixture may comprise as constituent f) further customary auxiliaries in amounts of from 0 to 15% by weight, preferred from 0 to 10% by weight, more preferred from 0 to 5% by weight. Suitable further customary auxiliaries are selected from flowing agents, acidifying agents, sweeteners, aromas, taste enhancers, thickeners and/or surfactants.

The invention also relates to a process for making granulates from a powder mixture, comprising as constituent at least a filler or at least an active ingredient or both. Whereby the powder mixture to be granulated may also contain binders and/or disintegrants and optionally further customary auxiliaries and the granulation is carried out with mixing by adding a granulation aid, which is Magnesium diacetate×4H₂O and/or Magnesium dichloride×6H₂O and/or Magnesium sulfate×7H₂O and/or Disodium sulfate×10H₂O and/or Sodium thiosulfate×5H₂O and/or Na₃PO₄×12 H₂O and/or Na₂HPO₄×12H₂O/7H₂O and/or Sodium acetate×3H₂O, granulating the powder mixture raising the temperature of the mixture to 30° C. or more and inducing the release of at least a part of the crystal water of the Magnesium diacetate×4H₂O and/or Magnesium dichloride×6H₂O and/or Magnesium sulfate×7H₂O and/or Disodium sulfate×10H₂O and/or Sodium thiosulfate×5H₂O and/or Na₃PO₄×12 H₂O and/or Na₂HPO₄×12H₂O/7H₂O and/or Sodium acetate×3H₂O. Preferred the temperature of the powder mixture is raised to a temperature of between 35° C. and 120° C., most preferred to a temperature of between 40° C. and 100° C. The thermal heat for the temperature enhancement is generated by a heating device.

According to the invention all forms of heating devices can be used, preferred are microwave radiation sources, infrared heater, infrared dryer, high shear mixer and extruder, whereby twin-screw extruders are most preferred.

The invention is further illustrated by the following FIGURES and examples

### FIGURES

FIG. 1: standard screw configuration of the extruder

### EXAMPLES

### Analytical Methods

Particle size distribution (d (0.1), d (0.5), d (0.9)) of the constituents of the powder mixture and produced granulate were determined using a Malvern Mastersizer 2000 (F.A. Malvern Panalytical). The granulates were measured in the dry powder form at 1.0-2.0 bar dispersion air pressure for about 30 to 60 seconds.

### Granulation Using an Extruder

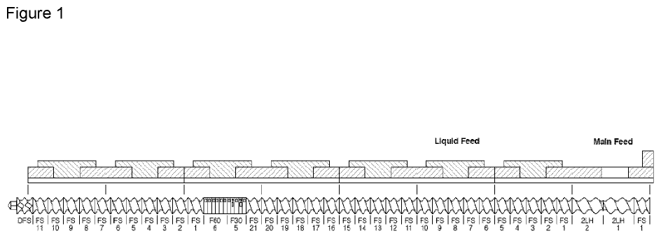

For the granulation of the powder mixture the respective constituents were weighed and shortly mixed by hand. This premix was sieved over 800 μm steel sieve and after that mixed for 10 minutes using a tumble blender, obtaining what is called in the following “Raw material (mixture)”. The Raw material (mixture) was granulated using a twin-screw extruder (Thermo Fisher Scientific, 11 mm) with following parameters (barrel L/D ratio of 40:1) and screw configuration as shown in FIG. 1:

| | | | Most preferred |
|---|---|---|---|
| Parameter | Range | Preferred range | range |
| Main process tem- | 30-150° C. | 35-120° C. | 40-100° C. |
| perature [° C.] | | | |
| Rotation speed [rpm] | 0-600 | 50-250 | 75-150 |
| Throughput [g/h] | 100-1500 | 150-900 | 250-600 |

The granulate leaving the extruder is directly processed further by a milling process or more preferred by a sieving process to display a good uniformity in the particle size distribution to obtain a granulate with a particle size particularly suitable for tableting.

### Tableting:

The preparation sieving of the granulates were performed with a vibratory sieve shaker (Retsch-AS 200) with the mesh size of 1000 μm for about 2 minutes with an amplitude of 1.0 mm/g.

The sieved fraction<1000 μm were mixed with additional 1.0% sodium stearyl fumarate in a tumble blender for about 2 minutes. This blend was further used for tableting.

The tableting was conducted in a multifunctional R&D press (Medelpharm-Styl'one Evo) with 10 mm biplane punches. The compaction pressure varied from 50 MPa to 400 MPa.

The tablets were analyzed (10 tablets per trial) in a semi-automatic tablet hardness tester (Sotax-SmartTest50). The tensile strength can be calculated according to USP<1217>.

TABLE 1
Examples demonstrating which energy input is needed to trigger the water release of
Magnesium diacetate × 4H₂O and/or
Magnesium dichloride × 6H₂O and/or Magnesium sulfate × 7H₂O
and/or Disodium sulfate × 10 H₂O and/or
Sodium thiosulfate × 5H₂O and/or Na₃PO₄ × 12 H₂O and/or
Na₂HPO₄ × 12H₂O/7H₂O and/or Sodium acetate × 3H₂O

| | | | | | | D 4,3 |
|---|---|---|---|---|---|---|
| | | | | | | [μm] |
| | | | d (0.1) | d (0.5) | d (0.9) | Volume mean |
| Trial | Filler/powder mixture | | [μm] | [μm] | [μm] | diameter |
| 1.1 | 100% Granulac 140 | Raw material | 7 | 57 | 148 | 68 |
| 1.2 | 85% Granulac 140, | screw configuration | 138 | 547 | 1272 | 631 |
| | 5% Kollidon 30, | (FIG. 1), | | | | |
| | 10% Na2SO4 × 10 H2O | 45° C. | | | | |
| 1.3 | 85% Granulac 140, | screw configuration | 175 | 534 | 1251 | 631 |
| | 5% Kollidon 30, | (FIG. 1), | | | | |
| | 10% NaHPO4 × 12 H2O | 80° C. | | | | |
| 1.4 | 85% Granulac 140, | screw configuration | 211 | 652 | 1312 | 714 |
| | 10% Na3PO4 × 12 H2O, | (FIG. 1), | | | | |
| | 5% Kollidon 30 | 55° C. | | | | |
| 1.5 | 80% Granulac 140, | screw | 213 | 559 | 1185 | 637 |
| | 15% Na2S2O3 × 5 H2O, | configuration (FIG. 1), | | | | |
| | 5% Kollidon 30 | 55° C. | | | | |
| 1.6 | 80% Granulac 140, | screw | 30 | 96 | 384 | 166 |
| | 15% NaAc × 3 H2O, | configuration (FIG. 1), | | | | |
| | 5% Kollidon 30 | 52° C. | | | | |
| 1.7 | 80% Granulac 140, | screw | 180 | 557 | 1165 | 624 |
| | 15% MgSO4 × 7 H2O, | configuration (FIG. 1), | | | | |
| | 5% Kollidon 30 | 100° C. | | | | |
| 1.8 | 80% Granulac 140, | screw | 118 | 398 | 1139 | 527 |
| | 15% MgAc × 4 H2O, | configuration (FIG. 1), | | | | |
| | 5% Kollidon 30 | 50° C. | | | | |
| 1.9 | 80% Granulac 140, | screw configuration | particles were too large to measure | | | |
| | 15% MgCl2 × 6 H2O, | (FIG. 1), | | | | |
| | 5% Kollidon 30 | 30° C. | | | | |

¹% means in table 1 and in the following % by weight.

TABLE 1
Examples demonstrating which energy input is needed to trigger the water release of
Magnesium diacetate × 4H₂O and/or
Magnesium dichloride × 6H₂O and/or Magnesium sulfate × 7H₂O
and/or Disodium sulfate × 10 H₂O and/or
Sodium thiosulfate × 5H₂O and/or Na₃PO₄ × 12H₂O and/or
Na₂HPO₄ × 12H₂O/7H₂O and/or Sodium acetate × 3H₂O

| | | | | | | D 4,3 |
|---|---|---|---|---|---|---|
| | | | | | | [μm] Volume |
| | | | d (0.1) | d (0.5) | d (0.9) | mean |
| Trial | Filler/powder mixture | | [μm] | [μm] | [μm] | diameter |
| 1.1 | 100% Granulac 140 | Raw material | 7 | 57 | 148 | 68 |
| 1.2 | 85% Granulac 140, | screw | 138 | 547 | 1272 | 631 |
| | 5% Kollidon 30, | configuration (FIG. 1), | | | | |
| | 10% Na2SO₄ × 10 H₂O | 45° C. | | | | |
| 1.3 | 85% Granulac 140, | screw | 175 | 534 | 1251 | 631 |
| | 5% Kollidon 30, | configuration (FIG. 1), | | | | |
| | 10% NaHPO₄ × 12 H₂O | 80° C. | | | | |
| 1.4 | 85% Granulac 140, | screw | 211 | 652 | 1312 | 714 |
| | 10% Na₃PO₄ × 12 H₂O, | configuration (FIG. 1), | | | | |
| | 5% Kollidon 30 | 55° C. | | | | |
| 1.5 | 80% Granulac 140, | screw | 213 | 559 | 1185 | 637 |
| | 15% Na₂S₂O₃ × 5 H2O, | configuration (FIG. 1), | | | | |
| | 5% Kollidon 30 | 55° C. | | | | |
| 1.6 | 80% Granulac 140, | screw | 30 | 96 | 384 | 166 |
| | 15% NaAc × 3 H₂O, | configuration (FIG. 1), | | | | |
| | 5% Kollidon 30 | 52° C. | | | | |
| 1.7 | 80% Granulac 140, | screw | 180 | 557 | 1165 | 624 |
| | 15% MgSO₄ × 7 H₂O, | configuration (FIG. 1), | | | | |
| | 5% Kollidon 30 | 100° C. | | | | |
| 1.8 | 80% Granulac 140, | screw | 118 | 398 | 1139 | 527 |
| | 15% MgAc × 4 H₂O, | configuration (FIG. 1), | | | | |
| | 5% Kollidon 30 | 50° C. | | | | |
| 1.9 | 80% Granulac 140, | screw | particles were too large to measure | | | |
| | 15% MgCl₂ × 6 H₂O, | configuration (FIG. 1), | | | | |
| | 5% Kollidon 30 | 30° C. | | | | |

¹% means in table 1 and in the following % by weight.

TABLE 2
Tableted granulates according to table 1

| | | | | | |
|---|---|---|---|---|---|
| compression | tensile | | | | |
| pressure | strength | | thickness | diameter | ejection |
| [MPa] | [N/mm²] | mass [mg] | [mm] | [mm] | force [N] |

Granulation trial 1.2 tableted

| | | | | | |
|---|---|---|---|---|---|
| 59.0 | 0.9 | 409.9 | 3.7 | 10.0 | 160 |
| 119.8 | 1.0 | 407.1 | 3.6 | 10.0 | 68 |
| 178.1 | 0.7 | 403.5 | 3.6 | 10.0 | 142 |
| 233.6 | 0.6 | 402.6 | 3.6 | 10.0 | 185 |

Granulation trial 1.3tableted

| | | | | | |
|---|---|---|---|---|---|
| 59.1 | 1.2 | 412.0 | 4.2 | 10.0 | 217 |
| 117.8 | 2.5 | 418.5 | 3.9 | 10.0 | 378 |
| 176.1 | 3.7 | 423.3 | 3.7 | 10.0 | 369 |
| 235.2 | 4.5 | 423.5 | 3.7 | 10.0 | 357 |

Granulation trial 1.4 Tableted

| | | | | | |
|---|---|---|---|---|---|
| 61.2 | 1.3 | 394.8 | 4.1 | 10.0 | 82 |
| 121.0 | 3.5 | 403.9 | 3.7 | 10.0 | 90 |
| 181.3 | 5.2 | 408.1 | 3.6 | 10.0 | 78 |
| 240.9 | 5.9 | 411.6 | 3.5 | 10.0 | 56 |

Granulation trial 1.5 Tableted

| | | | | | |
|---|---|---|---|---|---|
| 60.0 | 0.7 | 401.6 | 4.1 | 10.0 | 695 |
| 116.0 | 1.8 | 410.7 | 3.9 | 10.0 | 1299 |
| 171.1 | 3.1 | 418.0 | 3.8 | 10.0 | 1528 |
| 228.2 | 4.0 | 422.8 | 3.7 | 10.0 | 1515 |

Granulation trial 1.6 Tableted

| | | | | | |
|---|---|---|---|---|---|
| 60.7 | 0.7 | 405.8 | 4.3 | 10.0 | 142 |
| 119.9 | 1.9 | 406.9 | 3.9 | 10.0 | 257 |
| 176.5 | 2.9 | 411.5 | 3.8 | 10.0 | 293 |
| 232.6 | 3.7 | 412.3 | 3.7 | 10.0 | 298 |

Granulation trial 1.7 tableted

| | | | | | |
|---|---|---|---|---|---|
| 60.6 | 1.0 | 404.3 | 4.2 | 10.0 | 158 |
| 119.9 | 2.3 | 406.3 | 3.9 | 10.0 | 275 |
| 177.1 | 3.5 | 410.8 | 3.7 | 10.0 | 343 |
| 235.8 | 4.6 | 413.1 | 3.6 | 10.0 | 368 |

Granulation trial 1.9 tableted

| | | | | | |
|---|---|---|---|---|---|
| 61.1 | 1.5 | 407 | 4.1 | 10 | 39 |
| 121.3 | 3.1 | 416.1 | 3.8 | 10 | 46 |
| 180 | 4.2 | 427.8 | 3.8 | 10 | 52 |
| 237.1 | 4.7 | 434.3 | 3.8 | 10 | 51 |

1. A powder mixture for making granulates comprising:
a. 0-98.5% by weight of at least one filler,
b. 0-15% by weight of at least one binder,
c. 1.5-30% by weight of Magnesium diacetate×4H₂O and/or Magnesium dichloride×6H₂O and/or Magnesium sulfate×7H₂O and/or Disodium sulfate×10H₂O and/or Sodium thiosulfate×5H₂O and/or Na₃PO₄×12 H₂O and/or Na₂HPO₄×12H₂O/7H₂O and/or Sodium acetate×3H₂O,
d. 0-12.5% by weight of at least one disintegrant
e. 0-98.5% by weight of at least one active ingredient
a total of constituents a. to e being 100% by weight.
or
a powder mixture for making granulates comprising
a. 0-98.5% by weight of at least one filler,
b. 0-15% by weight of at least one binder,
c. 1.5-30% by weight of Magnesium diacetate×4H₂O and/or Magnesium dichloride×6H₂O and/or Magnesium sulfate×7H₂O and/or Disodium sulfate×10H₂O and/or Sodium thiosulfate×5H₂O and/or Na₃PO₄×12 H₂O and/or Na₂HPO₄×12H₂O/7H₂O and/or Sodium acetate×3H₂O,
d. 0-12.5% by weight of at least one disintegrant
e. 0-98.5% by weight of at least one active ingredient
f. 0-15% by weight further customary auxiliaries
a total of constituents a. to f being 100% by weight.

2. A powder mixture for making granulates comprising:
a. 7-97% by weight of at least one filler,
b. 1.5-10% by weight of at least one binder,
c. 1.5-25% by weight of Magnesium diacetate×4H₂O and/or Magnesium dichloride×6H₂O and/or Magnesium sulfate×7H₂O and/or Disodium sulfate×10H₂O and/or Sodium thiosulfate×5H₂O and/or Na₃PO₄×12 H₂O and/or Na₂HPO₄×12H₂O/7H₂O and/or Sodium acetate×3H₂O,
d. 0-10% by weight of at least one disintegrant
e. 0-90% by weight of at least one active ingredient
a total of constituents a. to e being 100% by weight or
a powder mixture for making granulates comprising
a. 7-97% by weight of at least one filler,
b. 1.5-10% by weight of at least one binder,
c. 1.5-25% by weight of Magnesium diacetate×4H₂O and/or Magnesium dichloride×6H₂O and/or Magnesium sulfate×7H₂O and/or Disodium sulfate×10H₂O and/or Sodium thiosulfate×5H₂O and/or Na₃PO₄×12 H₂O and/or Na₂HPO₄×12H₂O/7H₂O and/or Sodium acetate×3H₂O,
d. 0-10% by weight of at least one disintegrant
e. 0-98.5% by weight of at least one active ingredient
f. 0-10% by weight further customary auxiliaries
a total of constituents a. to f being 100% by weight.

3. A powder mixture for making granulates comprising:
a. 10-95% by weight of at least one filler,
b. 2.5-10% by weight of at least one binder,
c. 2.5-20% by weight of Magnesium diacetate×4H₂O and/or Magnesium dichloride×6H₂O and/or Magnesium sulfate×7H₂O and/or Disodium sulfate×10H₂O and/or Sodium thiosulfate×5H₂O and/or Na₃PO₄×12 H₂O and/or Na₂HPO₄×12H₂O/7H₂O and/or Sodium acetate×3H₂O,
d. 0-7.5% by weight of at least one disintegrant
e. 0-85% by weight of at least one active ingredient
a total of constituents a. to e being 100% by weight
or
a powder mixture for making granulates comprising
a. 10-95% by weight of at least one filler,
b. 2.5-15% by weight of at least one binder,
c. 2.5-20% by weight of Magnesium diacetate×4H₂O and/or Magnesium dichloride×6H₂O and/or Magnesium sulfate×7H₂O and/or Disodium sulfate×10H₂O and/or Sodium thiosulfate×5H₂O and/or Na₃PO₄×12 H₂O and/or Na₂HPO₄×12H₂O/7H₂O and/or Sodium acetate×3H₂O,
d. 0-7.5% by weight of at least one disintegrant
e. 0-85% by weight of at least one active ingredient
f. 0-15% by weight further customary auxiliaries
a total of constituents a. to f being 100% by weight.

4. The powder mixture according to claim 1, wherein a mean particle size d (0.5) of said Magnesium diacetate×4H₂O and/or Magnesium dichloride×6H₂O and/or Magnesium sulfate×7H₂O and/or Disodium sulfate×10H₂O and/or Sodium thiosulfate×5H₂O and/or Na₃PO₄×12 H₂O and/or Na₂HPO₄×12H₂O/7H₂O and/or Sodium acetate×3H₂O particles is less than 300 μm.

5. A process for making granulates from a powder mixture comprising at least a filler or at least an active ingredient or both, and optionally at least one binder and/or optionally at least one disintegrant and/or optionally at least one further customary auxiliary, comprising carrying out a granulation with mixing by adding a granulation aid, which is Magnesium diacetate×4H₂O and/or Magnesium dichloride×6H₂O and/or Magnesium sulfate×7H₂O and/or Disodium sulfate×10H₂O and/or Sodium thiosulfate×5H₂O and/or Na₃PO₄×12 H₂O and/or Na₂HPO₄×12H₂O/7H₂O and/or Sodium acetate×3H₂O, granulating the powder mixture by raising a temperature of the mixture to 50° C. or more and inducing the release of at least a part of the crystal water of the Magnesiumdiacetate×4H₂O and/or Magnesium dichloride×6H₂O and/or Magnesium sulfate×7H₂O and/or Disodium sulfate×10H₂O and/or Sodium thiosulfate×5H₂O and/or Na₃PO₄×12 H₂O and/or Na₂HPO₄×12H₂O/7H₂O and/or Sodium acetate×3H₂O.

6. The process according to claim 5, wherein the temperature of the powder mixture to be granulated is raised to between 35 and 120° C.

7. The process according to claim 6, wherein the temperature of the powder mixture is raised by a heating device.

8. The process according to claim 7, wherein the heating device is a microwave radiation source or an IR-Dryer or an IR-heater or a high shear mixer or an extruder or a combination thereof

9. The process according to claim 8, wherein the heating device is an extruder.

10. The process according to claim 5, wherein the granulate produced is directly milled or sieved.

11. The process according to claim 5, wherein the granulates are produced continuously.

12. A granulate obtained by a process according to any of claim 5 for tableting or filling capsules.

13. A food or feed, household cleaners, cleaning agents, laundry detergents, dishing washing tablets, or cosmetical, or agricultural, or medical or pharmaceutical preparations or mixtures thereof comprising a granulate prepared according to claim 5.

14. A granulation aid comprising diacetate×4H₂O and/or Magnesium dichloride×6H₂O and/or Magnesium sulfate×7H₂O and/or Disodium sulfate×10H₂O and/or Sodium thiosulfate×5H₂O and/or Na₃PO₄×12 H₂O and/or Na₂HPO₄×12H₂O/7H₂O and/or Sodium acetate×3H₂O as in a powder mixture.
